(19) United States
(12) Patent Application Publication
(10) Pub. No.: US 2005/0171610 A1
(43) Pub. Date: Aug. 4, 2005

(54) Mobile bearing spinal device and method

(75) Inventors: Steven C. Humphreys, Chattanooga, TN (US); Scott D. Hodges, Ooltewah, TN (US); Marc M. Peterman, Memphis, TN (US)

Correspondence Address: HAYNES AND BOONE, LLP, 901 MAIN STREET, SUITE 3100, DALLAS, TX, 75202, US

(73) Assignee: SDGI Holdings, Inc., Wilmington, DE (US)

(21) Appl. No.: 11/031,783

(22) Filed: Jan. 7, 2005

Related U.S. Application Data
(60) Provisional application No. 60/534,960, filed on Jan. 9, 2004.

(51) Int. Cl. A61F 2/44

(52) U.S. Cl. 623/17.15

(57) ABSTRACT

An artificial vertebral joint for interposition between a superior vertebra and an inferior vertebra, the artificial vertebral joint comprises a superior retaining portion and an inferior retaining portion. The joint further comprises a half-cylinder shaped mobile bearing adapted for insertion between the superior retaining portion and the inferior retaining portion, wherein the half-cylinder shaped mobile bearing is further adapted to move within the inferior retaining portion.

### CROSS-REFERENCE

This application claims priority from U.S. Provisional Patent Application Ser. No. 60/534,960 filed on Jan. 9, 2004, entitled “Posterior Lumbar Arthroplasty.” The following applications also claim priority to the above referenced provisional application and are related to the present application. They are incorporated by reference herein.
U.S. Utility patent application Ser. No. (Attorney Docket No. PC1146), filed on Jan. 7, 2005 and entitled “Spinal Arthroplasty Device and Method;”
U.S. Utility patent application Ser. No. (Attorney Docket No. P21769), filed on Jan. 7, 2005 and entitled “Dual Articulating Spinal Device and Method;”
U.S. Utility patent application Ser. No. (Attorney Docket No. P21756), filed on Jan. 7, 2005 and entitled “Split Spinal Device and Method;”
U.S. Utility patent application Ser. No. (Attorney Docket No. P21752), filed on Jan. 7, 2005 and entitled “Interconnected Spinal Device and Method;”
U.S. Utility patent application Ser. No. (Attorney Docket No. P21743), filed on Jan. 7, 2005 and entitled “Support Structure Device and Method;”
U.S. Utility patent application Ser. No. (Attorney Docket No. P21765), filed on Jan. 7, 2005 and entitled “Centrally Articulating Spinal Device and Method;” and
U.S. Utility patent application Ser. No. (Attorney Docket No. P21751), filed on Jan. 7, 2005 and entitled “Posterior Spinal Device and Method.”

### TECHNICAL FIELD

Embodiments of the invention relate generally to devices and methods for accomplishing spinal surgery, and more particularly in some embodiments, to spinal arthroplasty devices capable of being placed posteriorally into the vertebral disc space. Various implementations of the invention are envisioned, including use in total spine arthroplasty replacing, via a posterior approach, both the disc and facet functions of a natural spinal joint.

### BACKGROUND

As is known the art, in the human anatomy, the spine is a generally flexible column that can take tensile and compressive loads, allows bending motion and provides a place of attachment for ribs, muscles and ligaments. Generally, the spine is divided into three sections: the cervical, the thoracic and the lumbar spine. FIG. 1 illustrates schematically the lumbar spinal 1 and the sacrum regions 3 of a healthy, human spinal column. The sections of the spine are made up of individual bones called vertebrae and the vertebrae are separated by intervertebral discs which are situated therebetween.

FIG. 2 illustrates a portion of the right side of a lumbar spinal region with a healthy intervertebral disc 5 disposed between two adjacent vertebrae 7, 9. In any given joint, the top vertebra may be referred to as the superior vertebra and the bottom one as the inferior vertebra. Each vertebra comprises a generally cylindrical body 7a, 9a, which is the primary area of weight bearing, and three bony processes, e.g., 7b, 7c, 7d (two of which are visible in FIG. 2). As shown in FIG. 7A, in which all of the processes are visible, processes 7b, 7c, 7d extend outwardly from vertebrae body 7 at circumferentially spaced locations. The processes, among other functions, provide areas for muscle and ligament attachment. Neighboring vertebrae may move relative to each other via facet components 7e (FIG. 2), which extend from the cylindrical body of the vertebrae and are adapted to slide one over the other during bending to guide movement of the spine. There are two facet joints, each defined by upper and lower facet components, associated with adjacent vertebra. A healthy intervertebral disc is shown in FIG. 3. As shown in FIG. 3, an intervertebral disc has 4 regions: a nucleus pulposus 11, a transition zone 13, an inner annulus fibrosis region 15 and an outer annulus fibrosis 17. Generally, the inner annulus fibrosis region 15 and the outer annulus fibrosis region 17 are made up of layers of a fibrous gristly material firmly attached to the vertebral bodies above and below it. The nucleus pulposus 11 is typically more hydrated in nature.

These intervertebral discs function as shock absorbers and as joints. They are designed to absorb the compressive and tensile loads to which the spinal column may be subjected while at the same time allowing adjacent vertebral bodies to move relative to each other a limited amount, particularly during bending (flexure) of the spine. Thus, the intervertebral discs are under constant muscular and/or gravitational pressure and generally are the first parts of the lumbar spine to show signs of “wear and tear”.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis (i.e., spine fusion), which has been performed both anteriorally and/or posteriorally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be advantageous. It is also known to surgically replace a degenerative disc or facet joint with an artificial disc or an artificial facet joint, respectively. However, none of the known devices or methods provide the advantages of the embodiments of the present disclosure.

Accordingly, the foregoing shows there is a need for an improved spinal arthroplasty that avoids the drawbacks and disadvantages of the known implants and surgical techniques.

### SUMMARY

In one embodiment, an artificial vertebral joint for interposition between a superior vertebra and an inferior vertebra, the artificial vertebral joint comprises a superior retaining portion and an inferior retaining portion. The joint further comprises a half-cylinder shaped mobile bearing adapted for insertion between the superior retaining portion and the inferior retaining portion, wherein the half-cylinder shaped mobile bearing is further adapted to move within the inferior retaining portion.

In a second embodiment, an artificial vertebral joint is adapted for interposition between a superior vertebra and an inferior vertebra. The artificial vertebral joint comprises a first arthroplasty half comprising a first superior retaining portion, a first inferior retaining portion, and a first half-cylinder shaped mobile bearing adapted for insertion between the first superior retaining portion and the first inferior retaining portion. The first half-cylinder shaped mobile bearing is movable within the first inferior retaining portion. The artificial vertebral joint further comprises a second arthroplasty half comprising a second superior retaining portion, a second inferior retaining portion, and a second half-cylinder shaped mobile bearing adapted for insertion between the second superior retaining portion and the second inferior retaining portion. The second half-cylinder shaped mobile bearing is movable within the second inferior retaining portion.

In a third embodiment, a method of implanting an artificial spinal joint comprises creating first exposure through a patient's back to access an intervertebral space and inserting at least a portion of the artificial spinal joint through the first exposure. The method further comprises positioning a first anterior joint portion of the artificial spinal joint in the intervertebral space. The first anterior joint portion comprises a first superior retaining portion, a first inferior retaining portion, and a first half-cylinder shaped mobile bearing positioned between the first superior retaining portion and the first inferior retaining portion. The first half-cylinder shaped mobile bearing is further adapted to move within the first inferior retaining portion.

The embodiments disclosed may be useful for degenerative changes of the lumbar spine, post-traumatic, discogenic, facet pain or spondylolisthesis, and/or to maintain motion in multiple levels of the lumbar spine.

Additional and alternative features, advantages, uses and embodiments are set forth in or will be apparent from the following description, drawings, and claims.

### BRIEF DESCRIPTION OF THE DRAWINGS

FIG. 1 is a side elevation schematic view of the lumbar spinal and the sacrum regions of a healthy, human spinal column.

FIG. 2 is a detailed perspective view showing a portion of the right side of the lumbar vertebrae shown in FIG. 1 with a healthy disc disposed between two vertebrae.

FIG. 3 is a top perspective view of the intervertebral disc shown in FIG. 2 illustrating the major portions of the disc.

FIG. 4 is a side exploded elevation view of a portion of a lumbar spine showing a first embodiment of an artificial intervertebral joint constructed according to the principles of the disclosure.

FIG. 5 is an anterior elevation view of a portion of a lumbar spine showing the superior, disc and inferior portions of the left and right halves of an assembled artificial intervertebral joint constructed according to the first embodiment of the disclosure.

FIG. 6 is a side elevation view of the right half of the artificial intervertebral joint shown in FIG. 5.

FIG. 7A is a transverse, bottom-up-view of a portion of a lumbar spine showing the superior portion of the artificial intervertebral joint illustrated in FIG. 4.

FIG. 7B is a transverse, top-down-view of a portion of a lumbar spine showing the inferior portion of the artificial intervertebral joint illustrated in FIG. 4.

FIG. 8 is a transverse, bottom-up-view of a portion of a lumbar spine showing a second embodiment of a superior portion of an artificial intervertebral joint in which pedicle screws are used to assist in implantation.

FIG. 9 is a transverse, top-down-view of a portion of a lumbar spine showing a second embodiment of an inferior portion of an artificial intervertebral joint in which pedicle screws are used to assist in implantation.

FIG. 10 is a lateral view of a portion of a lumbar spine showing the superior portion of the artificial intervertebral joint shown in FIG. 8 with one of the pedicle screws being visible.

FIG. 11 is a lateral view of a portion of a lumbar spine showing the inferior and integrated disc portions of an artificial integral intervertebral joint shown in FIG. 9 with one of the pedicle screws being visible.

FIG. 12 is a posterior view of a portion of a lumbar spine showing the superior portion of the artificial intervertebral joint shown in FIG. 8 with two pedicle screws being visible.

FIG. 13 is a posterior view of a portion of a lumbar spine showing the inferior portion of the artificial intervertebral joint shown in FIG. 9 with two pedicle screws being visible.

FIG. 14 is a side elevation view of a portion of a lumbar spine showing the second embodiment with pedicle screws in an assembled position.

FIG. 15 is a posterior view of a portion of a lumbar spine showing a third embodiment of the inferior, disc and superior portions of an artificial intervertebral joint in which tension bands are used.

FIG. 16 is a side elevation view of a portion of a lumbar spine showing the third embodiment in which tension bands are used in an assembled position.

FIG. 17 is a transverse, bottom-up-view of a portion of a lumbar spine showing the superior portion of a fourth embodiment of an artificial intervertebral joint constructed according to the principles of the disclosure in which the facet joints are not replaced.

FIG. 18 is a transverse, top-down-view of a portion of a lumbar spine showing the inferior portion of the fourth embodiment of an artificial intervertebral joint.

FIG. 19 is an exploded perspective view of another embodiment of the present disclosure.

FIG. 20 is an exploded perspective view of another embodiment of the present disclosure.

FIG. 21 is an exploded perspective view of another embodiment of the present disclosure.

FIG. 22 is an exploded perspective view of another embodiment of the present disclosure.

FIG. 23 is a cross-sectional view of another embodiment of the present disclosure.

FIG. 24 is a cross-sectional view of another embodiment of the present disclosure.

FIG. 25 is a cross-sectional view exploded perspective view of another embodiment of the present disclosure.

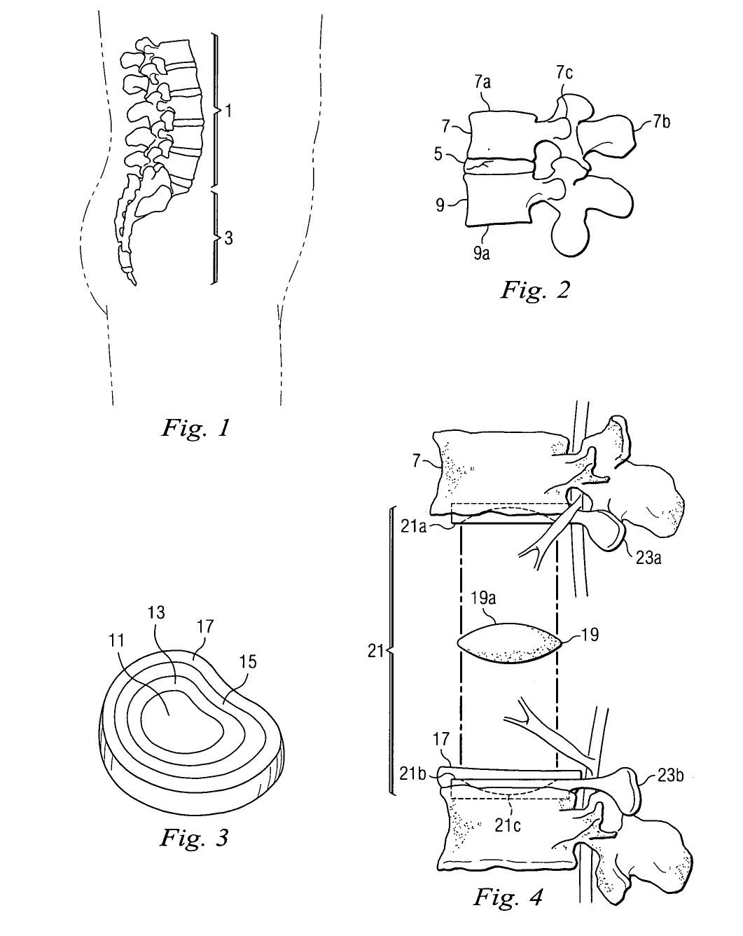

### DESCRIPTION

The drawings illustrate various embodiments of an artificial intervertebral joint for replacing an intervertebral disc or the combination of an intervertebral disc and at least one corresponding facet joint. Various embodiments of the artificial intervertebral joint according to the principles of the disclosure may be used for treating any of the problems that lend themselves to joint replacement including particularly, for example, degenerative changes of the lumbar spine, post-traumatic, discogenic, facet pain or spondylolisthesis and/or to maintain motion in multiple levels of the lumbar spine.

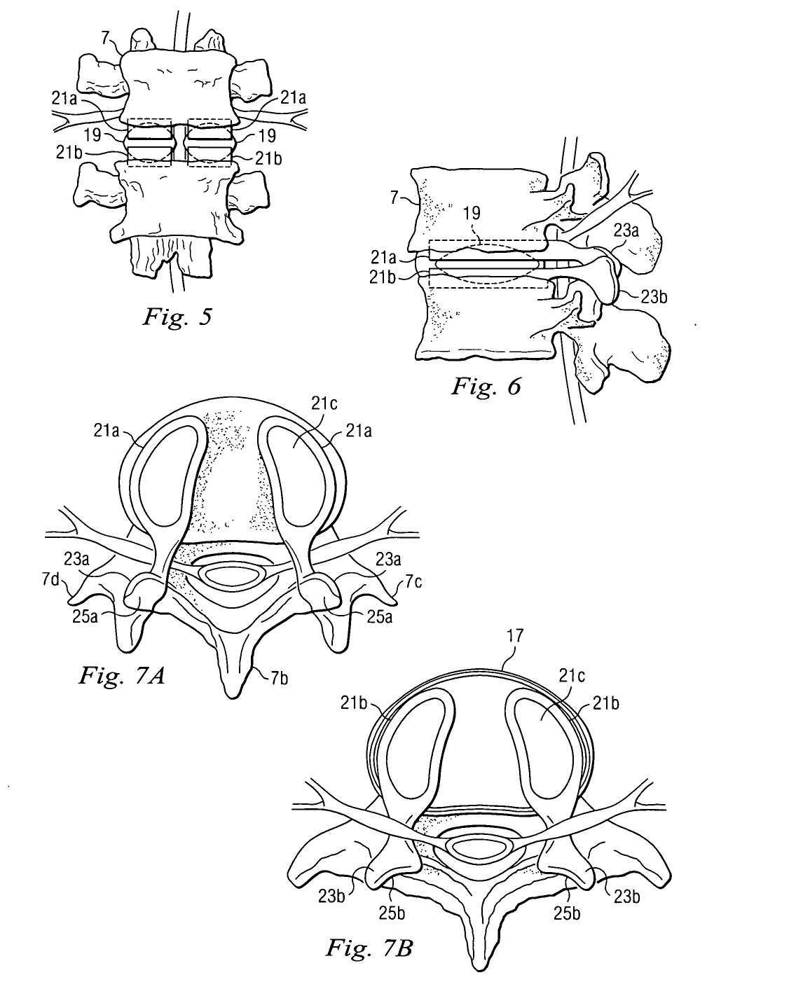

FIGS. 4-7 illustrate a first exemplary embodiment of an artificial intervertebral joint. As illustrated in FIGS. 4 and 5, each joint is composed of two arthroplasty halves, each of which has a spacer or disc 19 and a retaining portion 21. The retaining portion 21 includes a first retaining portion 21a and a second retaining portion 21b. In the example illustrated in FIG. 4, the first retaining portion 21a is superior to (above) the second retaining portion 21b and the disc 19 is situated therebetween. Although the artificial intervertebral joint according to this exemplary embodiment has two halves for each of the first retaining portion and the second retaining portion, it should be understood that alternative embodiments may be implemented such that the artificial intervertebral joint has a single first retaining member, a single second retaining member and a single spacer. It should also be understood that alternative embodiments may also be carried out with arthroplasties having a first retaining portion, a second retaining portion, and/or a disc which each consist of unequal sized halves or more than two components.

Further, as illustrated in FIG. 4, the first retaining portion 21a and the second retaining portion 21b are situated between two adjacent vertebrae. More particularly, the first retaining portion may be situated along an inferior surface of the upper of the two adjacent vertebrae and the second retaining portion may be situated above a superior surface of the lower of the two adjacent vertebrae. However, it should be understood by one of ordinary skill in the art that the first retaining portion and second retaining portion are not limited to such an arrangement, and may be oriented in different positions and/or shaped differently than what is illustrated herein.

The surfaces of the retaining portions 21a, 21b of the arthroplasty that contact the remaining end plates of the vertebrae may be coated with a beaded material or plasma sprayed to promote bony ingrowth and a firm connection therebetween. In particular, the surface to promote bone ingrowth may be a cobalt chromium molybdenum alloy with a titanium/calcium/phosphate double coating, a mesh surface, or any other effective surface finish. Alternatively or in combination, an adhesive or cement such as polymethylmethacrylate (PMMA) may be used to fix all or a portion of the implants to one or both of the endplates.

As discussed in more detail below, a significant portion of the outer annulus region 17 (see, e.g., FIGS. 4, 7B), in some embodiments about 300 degrees, may be retained on the inferior portion of the end plate, which acts as a stop retaining the lower retaining portions in place until bone ingrowth occurs to firmly attach the retaining portions to their respective vertebrae (FIG. 4 only shows a portion of the outer annulus 17 that is retained). In contrast, in conventional anterior arthroplasty about 270 degrees of the outer annulus region 17 typically is removed. In addition, pedicle screws may also be used for immediate fixation as described in more detail in connection with other embodiments discussed below.

In the various embodiments of this disclosure, the first retaining portion 21a and the second retaining portion 21b are structured so as to retain the disc 19 therebetween. For example, in the case of a disc 19 with two convex surfaces 19a, each of the first retaining portion 21a and the second retaining portion 21b may have a concave surface 21c which defines a space within which the disc 19 may be retained. For example, in the exemplary embodiment shown in FIG. 4, the upper convex surface 19a of the disc 19 fits within the concavity defined by the concave surface 21c of the first retaining portion 21a and the lower convex surface 19b of the disc 19 fits within the concavity defined by the concave surface 21c of the second retaining portion 21b.

FIG. 5 illustrates an anterior view of an exemplary assembled artificial intervertebral joint with both arthroplasty halves in place, and FIG. 6 shows a side view of the assembled artificial intervertebral joint shown in FIG. 5. As illustrated in FIGS. 5 and 6, the disc 19 is retained between the first retaining portion 21a and the second retaining portion 21b. It should be understood that although the disc 19 may be held between the first retaining portion 21a and the second retaining portion 21b, the disc 19 is free to slidably move within the space defined by the corresponding surfaces 21a of the first retaining portion 21a and the second retaining portion 21b. In this manner, limited movement between the adjacent vertebrae is provided.

In the exemplary embodiment illustrated in FIGS. 4, 5 and 6, the disc 19 is a separate component which is inserted between the first retaining portion 21a and the second retaining portion 21b. However, as discussed below, it should be understood that the spacer or disc 19 may be integrally formed with or integrated into in one or both of the first retaining portion 21a and the second retaining portion 21b.

In the exemplary embodiment of the disclosure, as illustrated best in FIGS. 4, 6, 7A and 7B, each of the retaining portions of the artificial intervertebral joint includes a first artificial facet component 23a and a second artificial facet component 23b. As shown in FIGS. 7A and 7B, the first artificial facet component 23a has a face 25a and the corresponding second artificial facet component 23b has a face 25b configured such that the face 25a matingly fits with the face 25b to stabilize adjacent vertebrae while preserving and guiding the mobility of each vertebrae with respect to the other vertebrae. Each set of the upper and lower retaining portions 21a, 21b may have a pair of facet components 23a, 23b, which together define a facet joint. For a total joint replacement with facets according to this embodiment, the left and right arthroplasties would define two adjacent facet joints when viewed from the posterior.

Regardless of whether artificial facet joints are provided, the respective upper and lower retaining portions associated with the left and right halves of the arthroplasty may be completely independent from the other. That is, as shown in FIG. 7A, for example, the first retaining portions 21a associated with each half are not in direct contact with each other. The same is true with respect to the second retaining portions 21 b shown in FIG. 7B. However, it should be understood by one of ordinary skill in the art that, even in the embodiment of the disclosure which includes artificial facet joints, at least a portion of the first retaining portions 21a of each half and/or at least a portion of the second retaining portions 21b of each half may directly contact and/or be connected to each other as described in more detail in connection with the discussion of FIGS. 17-18.

Further, in the various embodiments of the disclosure, the disc 19, the first retaining portion 21a and the second retaining portion 21b may be made of any appropriate material which will facilitate a connection that transmits compressive and tensile forces while providing for the aforementioned slidable motion in a generally transverse direction between each of the adjacent surfaces. For example, in the first embodiment, the first retaining portion 21a and the second retaining portion 21b may be typically made from any metal or metal alloy suitable for surgical implants such as stainless steel, titanium, and cobalt chromium, or composite materials such as carbon fiber, or a plastic material such as polyetheretherketone (PEEK) or any other suitable materials. The disc may be made from plastic such as high molecular weight polyethylene or PEEK, or from ceramics, metal, and natural or synthetic fibers such as, but not limited to, carbon fiber, rubber, or other suitable materials. Generally, to help maintain the sliding characteristic of the surfaces, the surfaces may be polished and/or coated to provide smooth surfaces. For example, if the surfaces are made of metal, the metal surfaces may be polished metal.

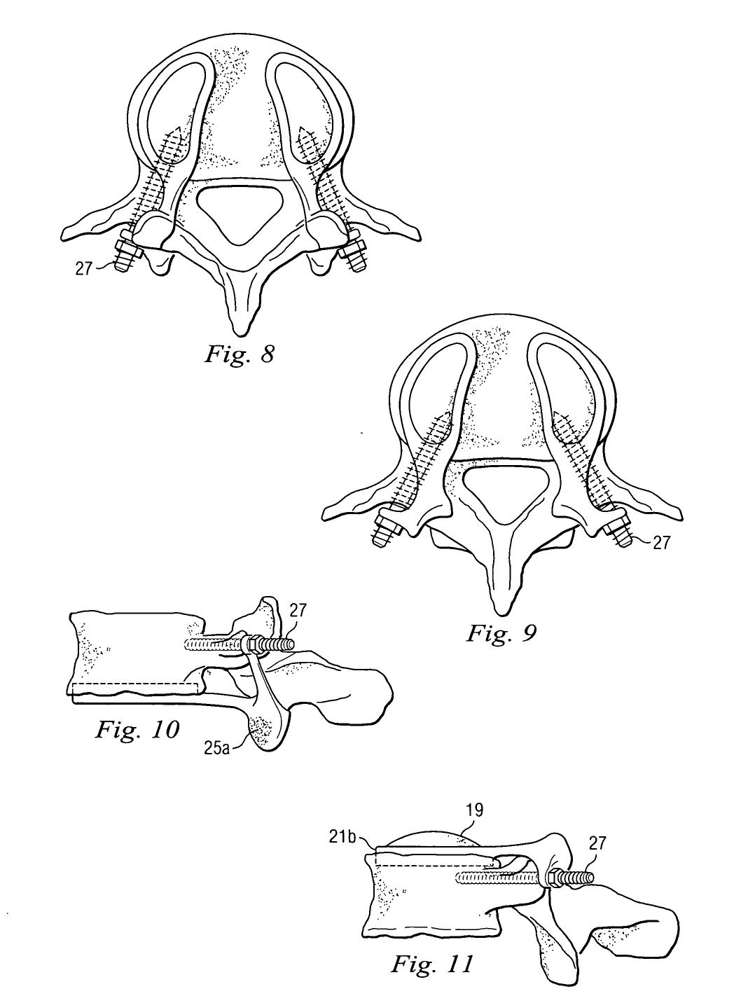

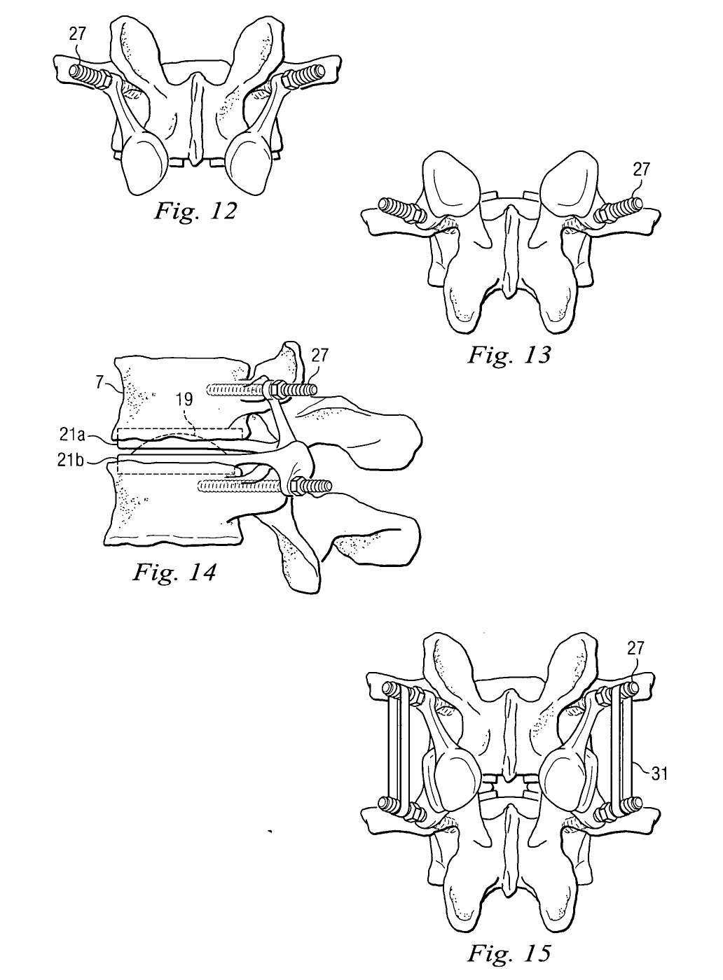

FIGS. 8-14 illustrate a second embodiment of an artificial intervertebral joint. Only features that differ from the first embodiment are discussed in detail herein. In the second exemplary embodiment, securing components, such as, for example, pedicle screws 27 are provided to provide a more secure and immediate connection between each of the first retaining portion 21a and/or the second retaining portion 21b to the corresponding vertebra. In addition, this embodiment illustrates a disc 19 which is integrated with one of the retaining portions, here lower retaining portion 21b. Disc 19 may be integrally formed from the same material as its retaining portion, but also may be separately formed from similar or dissimilar materials and permanently connected thereto to form an integral unit. In this embodiment, the disc 19 and the retaining portions may be all formed from metal.

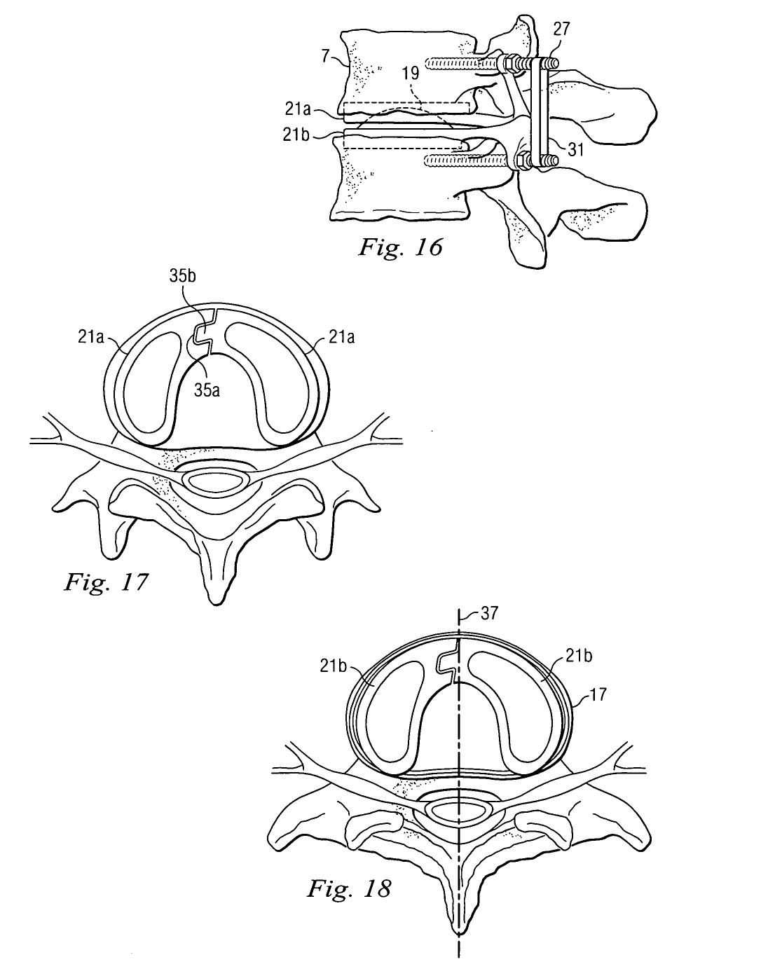

FIGS. 15 and 16 illustrate a third embodiment of an artificial intervertebral joint. In the third exemplary embodiment, additional securing components, such as, for example, tension bands 31 are provided to supplement or replace the function of posterior ligaments that limit the mobility between adjacent vertebrae by securing the first retaining portion 21a to the second retaining portion 21b. As shown in FIGS. 15-16, posterior tension bands 31 may be provided by wrapping them around the corresponding pedicle screws 27 or other convenient attachment points.

FIGS. 17 and 18 illustrate a fourth embodiment of an artificial intervertebral joint. In the exemplary embodiment illustrated in FIGS. 17 and 18, the artificial intervertebral joint may have all of the features discussed above except for artificial facet components. In this embodiment, the natural facet joints remain. The ligamentous tension band may also be left intact in some embodiments. In addition, this embodiment includes a specific example of an anterior midline connection between respective upper and lower retaining portions, which assists in maintaining the placement of the first retaining portion 21a and the second retaining portion 21b.

FIGS. 17 and 18 illustrate that it is possible to provide a first retaining portion 21a with a lock and key type pattern which is complemented by the corresponding mating portion provided on the second retaining portion 21b. More particularly, one half of the first retaining portion 21a has an outer boundary with a U-shaped portion 35a while the other half of the corresponding first retaining portion 21a has an outer boundary with a protruding portion 35b, which fits into the U-shaped portion 35a. As a result, each half of the first retaining portion 21a, 21b may be maintained in a predetermined position. However, the upper or lower retaining portions may fit together and/or be connected in the interbody space, e.g., near their midline anterior portions, in any manner that facilitates implantation and/or assists in providing and/or retaining the joint in a generally stable, symmetrical configuration. It may be even more important to provide such connection between the lower retaining portions due to the inward forces provided by annulus 17 remaining on the inferior end plate as shown in FIG. 18. A midline connection between the respective lower retaining portions will resist the force of the outer annulus tending to cause migration of the retaining portions toward the midline 37.

As shown in the various exemplary embodiments, other than the portions of the first and/or second retaining portions which may fit together like a lock and key to maintain the placement of the portions relative to each other, each half of the artificial intervertebral joint may be generally symmetrical about the midline 37 of the vertebrae.

Again, these exemplary embodiments are merely illustrative and are not meant to be an exhaustive list of all possible designs, implementations, modifications, and uses of the invention. Moreover, features described in connection with one embodiment of the disclosure may be used in conjunction with other embodiments, even if not explicitly stated above.

While it should be readily apparent to a skilled artisan from the discussion above, a brief description of a suitable surgical procedure that may be used to implant the artificial joint is provided below. Generally, as discussed above, the artificial intervertebral joint may be implanted into a body using a posterior transforaminal approach similar to the known TLIF or PLIF procedures. According to this approach, an incision, such as a midline incision, may be made in the patient's back and some or all of the affected disc and surrounding tissue may be removed via the foramina. Depending on whether any of the facet joints are being replaced, the natural facet joints may be trimmed to make room for the artificial facet joints. Then, the halves of the artificial intervertebral joint may be inserted piecewise through the left and right transforaminal openings, respectively. That is, the pieces of the artificial intervertebral joint including the upper and lower retaining portions, with or without facet components, and the artificial disc, if provided separately, fit through the foramina and are placed in the appropriate intervertebral space. The pieces of the artificial joint may be completely separated or two or more of them may be tied or packaged together prior to insertion through the foramina by cloth or other materials known in the art. In cases where at least a portion of the outer annulus of the natural disc can be retained, the lower retaining portions of each side of the artificial intervertebral joint are inserted such that they abut a corresponding portion of the annulus. If a midline anterior connection is provided, the left and right halves of the retaining members are fitted together and held in place by the outer annulus. As such, the remaining portion of the annulus may be in substantially the same place as it was prior to the procedure.

Further, in the cases where the annulus of the natural disc must be removed completely or this is insufficient annulus remaining, it is possible, for example, to use the embodiment of the disclosure where the pedicle screws are implemented so as to be assured that the pieces of the artificial intervertebral joint remain in place. It should be understood by one of ordinary skill in the art that the artificial joint could be implanted via an anterior approach or a combined anterior and posterior approach, although the advantages of a posterior procedure would be limited. For example, some of the pieces of the artificial intervertebral joint may be inserted from an anterior approach and others posteriorly. The anteriorly and posteriorly placed portions could be fitted together similar to the embodiment shown in FIGS. 17 and 18.

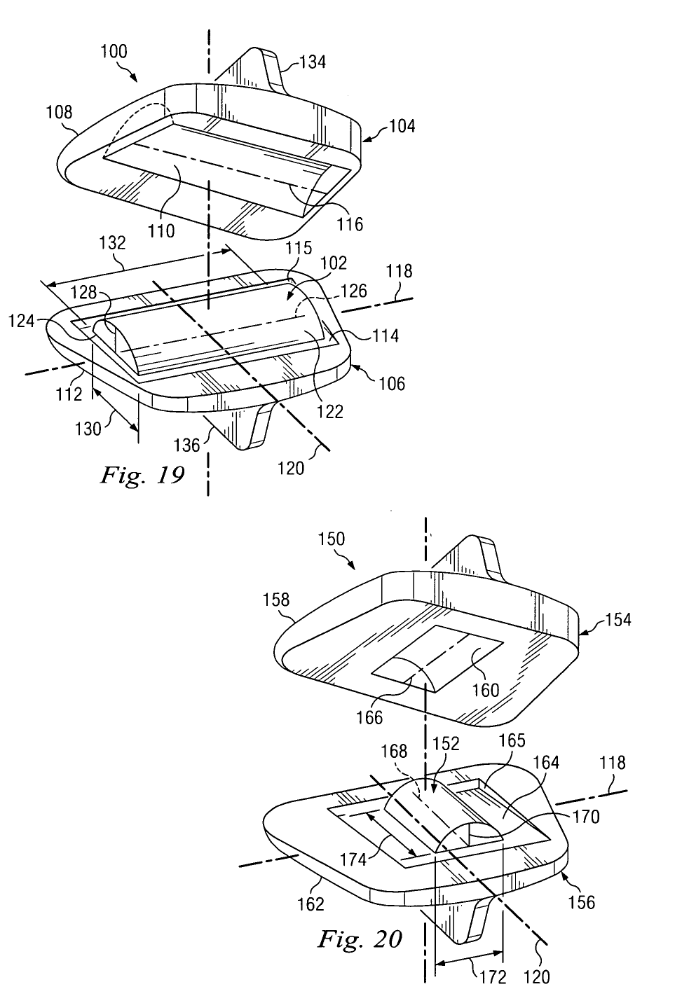

Referring now to FIG. 19, in this embodiment, an artificial intervertebral joint 100 may include a spacer or mobile bearing 102 interposed between two endplate assemblies 104, 106. The endplate assembly 104 may include an exterior surface 108 and a superior retaining portion 110. The endplate assembly 106 may include an exterior surface 112, an inferior retaining portion 114, and a motion stop surface 115. In this embodiment the retaining portion 110 may be a half or semi-cylindrical trough that permits smooth articulation with the spacer 102. The retaining portion 110 may have an elongated shape with a longitudinal axis 116 that is aligned approximately collinear or parallel with a transverse axis 118 of the assembled joint 100 and is centered about an anterior-posterior axis 120 extending through the assembled joint 100. The transverse axis 118 and the anterior-posterior axis 120 may extend through the intervertebral disc space between vertebrae 7, 9 when the joint 100 is installed.

The spacer 102 may be a half or semi-cylinder with a curved superior surface 122 and a flattened inferior surface 124. The spacer 102 may have an elongated shape with a longitudinal axis 126 that is aligned approximately parallel or collinear with the transverse axis 118 of the assembled joint 100. The spacer 102 may have a height dimension 128, a width dimension 130, and a length dimension 132. The half or semi-cylinder shape of the spacer 102 is broadly understood to include a variety of elongated shapes including bean shaped, ellipsoid, half cylinders with rounded edges, or half cylinders with curved superior and inferior surfaces. Although semi-spherical surfaces may also be employed, the more cylindrical shapes may be easier to manufacture.

The retaining portion 114 may be a tray that permits a smooth interaction with the spacer 102. In this embodiment the retaining portion 114 is flat to match the flattened inferior surface 124. The motion stop surface 115 may form a raised perimeter around the retaining portion 114. The retaining portion 114 may be slightly wider than the width dimension 130 to permit anterior-posterior translation of the spacer 102 with respect to the retaining portion 114. The retaining portion 114 may be slightly longer than the length dimension 132 to permit lateral translation of the spacer 102 with respect to the retaining portion 114. It is understood than in an alternative embodiment, the inferior retaining portion may be curved to match the shape of a curved inferior surface of a spacer. U.S. application Ser. No. 10/75,860 entitled “Mobile Bearing Articulating Disc” and filed Jan. 7, 2004 discloses sother articulating spacer embodiments and is incorporated by reference herein. U.S. application Ser. No. 10/806,961 entitled “Constrained Artificial Spinal Disc” and filed Mar. 23, 2004 also discloses other articulating spacer embodiments and is incorporated by reference herein.

It is understood that in alternative embodiments, movement of the spacer with respect to the inferior retaining portion may be controlled by the position of the motion stop surface and the resulting size of the flattened inferior surface. For example, if the inferior retaining portion is wider than the width dimension of the spacer but closely matches the length dimension, the motion of the spacer may be limited to anterior-posterior translation along the inferior retaining portion. If the inferior retaining portion matches the width dimension of the spacer but provides clearance along the length dimension, the motion of the spacer may be limited to lateral translation. If the inferior retaining portion is wider and longer than the flattened inferior surface, the spacer may be permitted to rotate or pivot on the inferior retaining portion.

The artificial intervertebral joint 100 may further comprise connection components 134, 136 which may be keels extending from the endplate assemblies 104, 106, respectively. The keels 134, 136 may engage the vertebral bodies 7, 9, respectively to secure the joint 100. It is understood that a variety of connection components may be used to secure the intervertebral joint in place. For example, other suitable connection components may include spikes, ridges, bone screws, and/or surface textures.

The joint 100 may be installed between the vertebral bodies 7, 9 using an anterior, posterior, transforaminal, or other approach known in the art. The curved superior surface 122 may be placed into articulating contact with the superior retaining portion 110 and the flattened inferior surface 124 may be placed into articulating contact with the inferior retaining portion 114, within the boundaries of the motion stop surface 115. In this embodiment, the retaining portion 114 provides clearance for both the width 130 and length 132 dimensions of the spacer 102, allowing the spacer to translate in both anterior-posterior and lateral directions and further allowing limited torsion in the joint 100. The curved superior surface 122, and the superior retaining portion 110 may articulate to permit flexion-extension motion at the joint 100. In this embodiment, lateral bending may be limited or precluded except for motions that decouple the spacer 102 from either of the endplate assemblies 104, 106 and create “lift-off.”

The spacer 102 and the two endplate assemblies 104, 106 may be formed of any suitable biocompatible material including metals such as cobalt-chromium alloys, titanium alloys, nickel titanium alloys, and/or stainless steel alloys. Ceramic materials such as aluminum oxide or alumnia, zirconium oxide or zirconia, compact of particulate diamond, and/or pyrolytic carbon may also be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. The spacer and endplate assemblies 104, 106 may be formed of different materials, thus permitting metal on metal, metal on ceramic, metal on polymer, ceramic on ceramic, ceramic on polymer, or polymer on polymer constructions. To create a smooth articulation between all contacting surfaces, the superior retaining portion, the inferior retaining portion, and at least some of the surfaces of the spacer may be ground and polished.

Exterior surfaces 108, 112 may include features or coatings which enhance the fixation of the implanted prosthesis. For example, the surfaces may be roughened such as by chemical etching, bead-blasting, sanding, grinding, serrating, and/or diamond-cutting. All or a portion of the bone contacting surfaces of the exterior surfaces 108, 112 may also be coated with a biocompatible and osteoconductive material such as hydroxyapatite (HA), tricalcium phosphate (TCP), and/or calcium carbonate to promote bone in growth and fixation. Alternatively, osteoinductive coatings, such as proteins from transforming growth factor (TGF) beta superfamily, or bone-morphogenic proteins, such as BMP2 or BMP7, may be used.

Referring now to FIG. 20, an artificial intervertebral joint 150 may be substantially similar to the joint 100 except for the differences described below. In this embodiment, the joint 150 may include a spacer 152 interposed between two endplate assemblies 154, 156. The endplate assembly 154 may include an exterior surface 158 and a superior retaining portion 160. The endplate assembly 156 may include an exterior surface 162, an inferior retaining portion 164, and a motion stop surface 165. In this embodiment the retaining portion 160 may be a half or semi-cylindrical trough that permits smooth articulation with the spacer 152. The retaining portion 160 may have an elongated shape with a longitudinal axis 166 that is aligned approximately collinear or parallel with the anterior-posterior axis 120 extending through the assembled joint 150. The spacer 152 may have an elongated shape with a longitudinal axis 168 that is aligned approximately parallel or collinear with the anterior-posterior axis 120. The spacer 152 may have a height dimension 170, a width dimension 172, and a length dimension 174.

The retaining portion 164 may be wider than the width dimension 172 to permit lateral translation of the spacer 152 with respect to the flattened inferior surface 164. The retaining portion 164 may be slightly longer than the length dimension 174 to permit anterior-posterior translation of the spacer 152 with respect to the flattened inferior surface 164. In this embodiment, the inferior retaining portion 164 provides clearance for both the width 172 and length 174 dimensions of the spacer 152, allowing the spacer to translate in both anterior-posterior and lateral directions and further allowing limited torsion in the joint 100. In this embodiment, the retaining portion 110 and the spacer 152 may articulate to permit lateral bending motion at the joint 100. With this orientation of the spacer 152, flexion-extension may be limited or precluded except for motions that decouple the spacer 152 from either of the endplate assemblies 154, 156 and create “lift-off.”

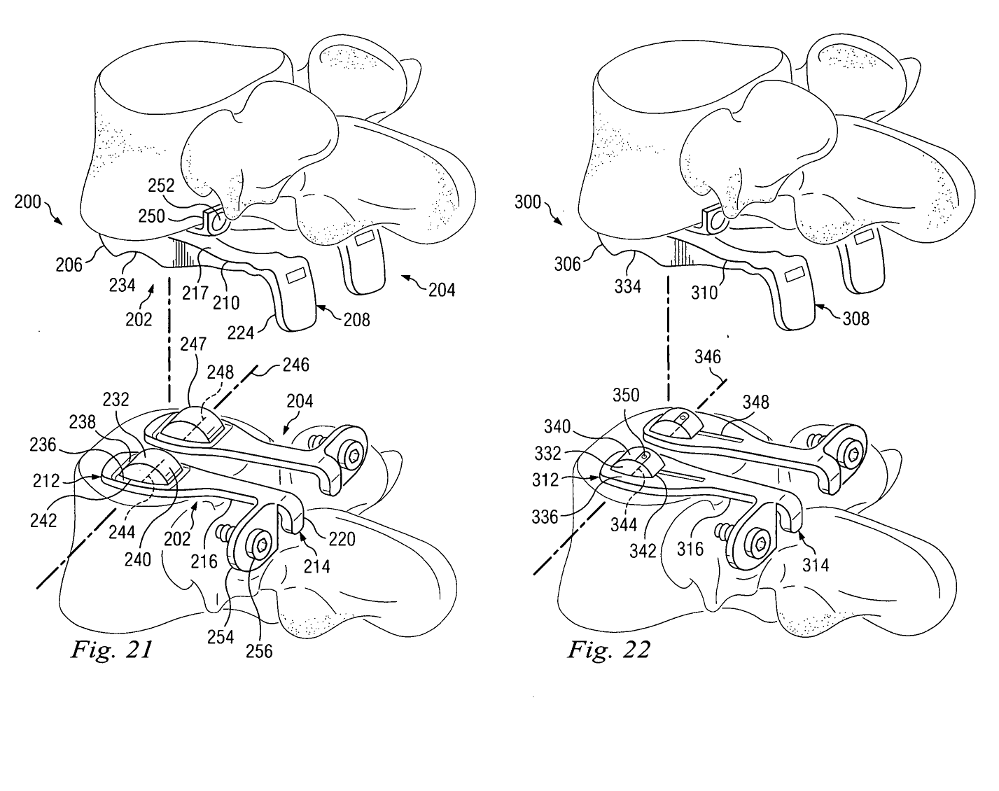

Referring now to FIG. 21, in this embodiment, an artificial intervertebral joint 200 may include two arthroplasty halves 202, 204 which may be inserted between the vertebrae 7, 9. The arthroplasty half 202 may include a rostral anterior joint component 206, a rostral posterior joint component 208, and a rostral bridge 210 extending between the anterior component 206 and the posterior component 208. The arthroplasty half 202 may further include a caudal anterior joint component 212, a caudal posterior joint component 214, and a caudal bridge 216 extending between the anterior component 212 and the posterior component 214. The arthroplasty half 204 may be substantially similar in structure and function to the arthroplasty half 202 and therefore will be described in only limited detail.

The terms “rostral” and “caudal” are used in some embodiments to describe the position of components of the embodiments. While rostral is typically used in the art to describe positions toward the head and caudal is used to describe positions toward the tail or foot, as used herein, rostral and caudal are used simply as modifiers for the relative locations of components of the illustrated embodiments. For example, rostral components may be on one side of an illustrated joint, and caudal may be on another side of the joint. Components labeled as rostral or caudal to describe an illustrated embodiment are not intended to limit the orientation of a device or application of a method relative to a patient's anatomy, or to limit the scope of claims to any device or method.

In this embodiment, the rostral bridge 210 may include a jog 217 to create an exit portal and an artificial foramen for the exiting nerve root. Also in this embodiment, the caudal posterior joint component 214 may include a posterior protrusion 220. Either of the bridges 210, 216, but particularly the caudal bridge 216, may be a “super” or artificial pedicle which may supplement or replace a natural pedicle.

In this embodiment, the arthroplasty half 202 may include a spacer 232 interposed between the rostral and caudal anterior joint components 206, 212. The rostral anterior joint component 206 may include a superior retaining portion 234. The caudal anterior joint component 212 may include an inferior retaining portion 236, and a motion stop surface 238. In this embodiment the retaining portion 234 may be a half or semi-cylindrical trough that permits smooth articulation with the spacer 232. The superior retaining portion 234 may be similar to the superior retaining portion 110 except for the differences described.

The spacer 232 may be a half or semi-cylinder with a curved superior surface 240 and a flattened inferior surface 242. The spacer 232 may be similar to the spacer 102 except for the differences described. The spacer 232 may include a longitudinal axis 244 that is aligned approximately parallel or collinear with a transverse axis 246 of the assembled joint 200.

The retaining portion 236 may be a tray that permits a smooth interaction with the spacer 232. The retaining portion 236 may be similar to the retaining portion 114 except for the differences described. In this embodiment, because the retaining portion 236 is larger than the inferior surface 242 in both dimensions, the retaining portion 236 may permit anterior-posterior and lateral translation of the spacer 232 with respect to the retaining portion 236. Further, rotation or pivoting between the spacer 232 and the retaining portion 236 may be permitted. As described above, the size of the retaining portion 236 and the location of the motion stop surface 238 may restrict or permit translation or rotation as desired.

The arthroplasty half 204 may be configured similar to arthroplasty half 202 except for the differences noted. Specifically, the arthroplasty half 204 may include a spacer 247 positioned and aligned similarly to the spacer 232. A longitudinal axis 248 of the spacer 232 may be aligned approximately parallel or collinear with the transverse axis 246 and also collinear with the longitudinal axis 244 of the spacer 232.

The rostral posterior joint component 208 may include a posterior socket 224 configured to engage the posterior protrusion 220. A radius of curvature for the posterior protrusion 220 may be smaller than a radius of curvature for the posterior socket 224, thereby permitting motion and limiting binding between the posterior joint components 208, 214. The radii of curvature for the posterior socket 224 and the posterior protrusion 220 may emanate from a common center of rotation for the arthroplasty half 202. In this embodiment, the radius of curvature for the posterior socket 224 is relatively large, and the resulting joint is loosely constrained. In an alternative embodiment, a tight radius of curvature for the posterior protrusion of the caudal posterior component matched with a rostral posterior component having a tight radius of curvature may create a tightly constrained posterior joint.

The size and shape of the anterior components 206, 212 and the bridge components 210, 216 may be limited by the constraints of a posterior surgical approach. For example, the anterior components 206, 212 may be configured to cover a maximum vertebral endplate area to dissipate loads and reduce subsidence while still fitting through the posterior surgical exposure, Kambin's triangle, and other neural elements. The width of the bridge components 210, 216 are also minimized to pass through Kambin's triangle and to co-exist with the neural elements.

The arthroplasty half 202 further includes features for affixing to the vertebrae 7, 9. It is understood, however, that in an alternative embodiment, the fixation features may be eliminated. Arthroplasty half 202 may include a connection component 250 extending rostrally from the rostral anterior joint component 206. The connection component 250 in this embodiment is an aperture adapted to receive a bone fastener such as screw 252. The orientation of the connection component 250 permits the screw 252 to affix to the cylindrical vertebral body 7a.

Arthroplasty half 202 may further include a connection component 254 attached to or integrally formed with the caudal posterior joint component 214. The connection component 254 in this embodiment is an aperture adapted to receive a bone fastener such as screw 256. The orientation of the connection component 254 permits the screw 256 to become inserted extrapedicularly such that the screw travels a path angled or skewed away from a central axis defined through a pedicle. Extrapedicular fixation may be any fixation into the pedicle that does not follow a path down a central axis defined generally posterior-anterior through the pedicle. In this embodiment, the screw passes through a lateral wall of the pedicle and may achieve strong cortical fixation. In all embodiments, the screws may be recessed so as not to interfere with articulations, soft tissues, and neural structures.

In an alternative embodiment, for example as shown in FIG. 14, a connection component extending from the posterior component 254 may be oriented to permit the screw to become inserted intrapedicularly such that the screw travels a path generally along the central axis through the pedicle. In still another alternative embodiment, the posterior connection component may connect to the generally cylindrical body portion 9a. It is understood that in other alternative embodiments, the connection components may extend at a variety of angles, in a variety of directions from the various components of the arthroplasty half. For example, a connection component may extend from the rostral bridge rather than the rostral anterior joint component.

As shown in FIG. 21, the rostral components 206, 208, 210 of the arthroplasty half 102 are integrally formed. It is understood that in a modular alternative embodiment, these components may be removably coupled to one another. For example, the rostral anterior joint component may be installed separate from the bridge. After the anterior component is in place, the bridge may be attached to the anterior component by any fastening mechanism known in the art, for example a threaded connection, a bolted connection, or a latched connection. A modular rostral posterior component may then be attached by a similar fastening mechanism to the bridge to complete the rostral portion of the arthroplasty half.

The artificial intervertebral joint 200 may be installed between the vertebrae 7, 9 as will be described below. Although installation will be described with respect to arthroplasty half 202, it is understood that the arthroplasty half 204 may be installed in a similar manner. Generally, as discussed above, the artificial intervertebral joint 200 may be implanted into a body using a posterior transforaminal approach similar to the known TLIF or PLIF procedures. PLIF approaches are generally more medial and rely on more retraction of the traversing root and dura to access the vertebral interspace. The space between these structures is known as Kambin's triangle. TLIF approaches are typically more oblique, requiring less retraction of the exiting root, and less epidural bleeding with less retraction of the traversing structures. It is also possible to access the interspace using a far lateral approach, above the position of the exiting nerve root and outside of Kambin's triangle. In some instances it is possible to access the interspace via the far lateral without resecting the facets. Furthermore, a direct lateral approach through the psoas is known. This approach avoids the posterior neural elements completely. Embodiments of the current invention are anticipate that could utilize any of these common approaches.

According to at least one of these approaches, an incision, such as a midline incision, may be made in the patient's back and some or all of the affected disc and surrounding tissue may be removed via the foramina. The superior endplate surface of the vertebra 9 may be milled, rasped, or otherwise resected to match the profile of the caudal anterior bone contacting surface, to normalize stress distributions on the superior endplate surface of the vertebra 9, and/or to provide initial fixation prior to bone ingrowth. The preparation of the endplate of vertebra 9 may result in a flattened surface or in surface contours such as pockets, grooves, or other contours that may match corresponding features on the bone contacting surface. The inferior endplate of the vertebra 7 may be similarly prepared to receive the rostral anterior joint component 206 to the extent allowed by the exiting nerve root and the dorsal root ganglia. Depending on whether any of the facet joints are being replaced, the natural facet joints of vertebrae 7, 9 may be trimmed to make room for the posterior components 208, 214.

The halves 202, 204 of the artificial intervertebral joint 200 may then be inserted piecewise through the left and right transforaminal openings, respectively. That is, the pieces of the artificial intervertebral joint 100 including the rostral and caudal anterior joint components 206, 212 respectively fit through the foramina and are placed in the appropriate intervertebral disc space between the generally cylindrical bodies 7a, 9a. The pieces of the artificial joint 200 may be completely separated or two or more of them may be tied or packaged together prior to insertion through the foramina by cloth or other materials known in the art. In cases where at least a portion of the outer annulus of the natural disc can be retained, the caudal anterior joint components of each side of the artificial intervertebral joint are inserted such that they abut a corresponding portion of the annulus. The bridges 210, 216 may extend posteriorly from the anterior joint components 206, 212 and posteriorly from the intervertebral disc space. The posterior components 208, 214 are positioned posteriorly of the intervertebral disc space to replace or supplement the function of the natural facet joints. The screw 252 may be inserted through the connection component 250 and into the generally cylindrical body 7a, and the screw 256 may be inserted through the connection component 254 and into adjacent bone such as the pedicle. It is understood that the screws may be implanted either after the entire arthroplasty half 202 has been implanted or after each of the rostral and caudal component has been implanted.

After installation, the spacer 232 may move in a similar way to the movement of the spacer 102, generally permitting flexion-extension motion, anterior-posterior translation, lateral translation, and limited torsion. As described above, any of these motions may be limited by limiting or increasing the clearance between the spacer 232 and the motion stop surface 238. The posterior joint, created by the rostral posterior joint component 208 and the caudal posterior joint component 214, allow the arthroplasty half 202 to resist shear forces, particularly anterior-posterior forces. Movement of the rostral anterior joint component 206 relative to the caudal anterior joint component 212 may be limited by the displacement of the posterior protrusion 220 within the posterior socket 224. For example, lateral translation of the rostral anterior joint component 206 relative to the caudal anterior joint component 212 may be limited by the posterior joint. Rotational motion about a longitudinal axis defined by the cylindrical bodies 7a, 9a may be limited both by the constraint in the posterior joint and by the combined constraint provided by the two arthroplasty halves 202, 204. Further, the posterior joint may restrict any true lateral bending degree of freedom.

Under certain conditions, the joint 200 may overcome these design restrictions to permit limited lateral, rotational, and coupled movements. For example, the anterior joint component 206 may become disconnected from the spacer 232 and experience limited “lift-off,” thereby permitting additional degrees of freedom and coupled motions beyond strict flexion-extension motion. The self-centering nature of the anterior joint may encourage reconnection and alignment after lift-off occurs. The limited disconnection of the anterior joint components may be accommodated by the degree of constraint in the posterior joint. For example, relatively loose constraint in the posterior joint permits greater amounts of lift-off. Some degree of constraint in the posterior joint may be useful, however, to encourage reconnection and alignment of the anterior joint.

In general, a simple, anteriorly located ball and socket joint which is tightly constrained with each component having the same or similar radii of curvature may allow flexion-extension, lateral bending, and torsion motions while resisting shear forces and limiting translation. By adding an additional highly constrained ball and socket joint to the posterior components, an additional degree of freedom may be limited, such as torsion. Additional joints may further limit degrees of freedom of motion. If the anterior or posterior joints are permitted to disconnect or disarticulate additional degrees of freedom may be permitted as described above. Changing the shape of or clearance between the ball and socket components will also permit additional degrees of motion.

The robust and forgiving structure of the anterior and posterior joints also permits misalignment and slight inaccuracy in the placement of the arthroplasty halves 202, 204. For example, the self-aligning structure of the anterior joint components 206, 212, 232 may tolerate a certain amount of misalignment between the components. Thus, the insertion trajectories for the components 206, 212 may be slightly misaligned. The interaction of the posterior protrusion 220 and the posterior socket 224 may also accommodate parallel misalignment and/or anterior-posterior misalignment between the arthroplasty halves 202, 204.

In an alternative embodiment, any of the artificial intervertebral joints described above may further include a rostral keel extending from the rostral anterior component and/or a caudal keel extending from the caudal anterior joint component and along the caudal bridge. The rostral keel may engage the inferior endplate of the vertebral body 7a, and the caudal keel may engage the superior endplate of the vertebral body 9a and a superior face of a pedicle of vertebra 9. It is understood that the inferior endplate of the body 7a may be milled or otherwise prepared to receive the rostral keel. Likewise, the superior endplate of the body 9a and the pedicle of vertebra 9 may be milled, chiseled, or otherwise prepared to create a channel for receiving the caudal keel. The keels may help to connect to the bone and limit movement of the arthroplasty half to the desired degrees to freedom. The keels may have an angled or semi-cylindrical cross section. It is understood that more than one keel may be used on any given component.

Referring now to FIGS. 22, in this embodiment, an artificial intervertebral joint 300 may include two arthroplasty halves 302, 304 which may be inserted between the vertebrae 7, 9. The arthroplasty halves 302, 304 may be similar to the arthroplasty halves 202, 204 except for the differences described. The arthroplasty half 302 may include a rostral anterior joint component 306, a rostral posterior joint component 308, and a rostral bridge 310 extending between the anterior component 306 and the posterior component 308. The arthroplasty half 302 may further include a caudal anterior joint component 312, a caudal posterior joint component 314, and a caudal bridge 316 extending between the anterior component 312 and the posterior component 314.

In this embodiment, the arthroplasty half 302 may include a spacer 332 interposed between the rostral and caudal anterior joint components 306, 312. The rostral anterior joint component 306 may include a superior retaining portion 334. The caudal anterior joint component 312 may include an inferior retaining portion 336. In this embodiment the retaining portion 334 may be a half or semi-cylindrical trough that permits smooth articulation with the spacer 332. The spacer 332 may be a half or semi-cylinder with a curved superior surface 340 and a flattened inferior surface 342. The spacer 332 may include a longitudinal axis 344 that is aligned approximately parallel or collinear with the transverse axis 346 of the assembled joint 300.

The retaining portion 336 may be a flat surface that permits a smooth interaction with the spacer 332. In this embodiment, the retaining portion 336 further comprises a track or groove 348 that extends in a generally anterior-posterior direction when installed. A pin 350 may extend through the spacer 332 and movably attach to the track 348 to permit the spacer 332 to move along the track in an anterior-posterior direction. The spacer 332 may also be permitted to pivot about the pin 350. Lateral translation may be limited or restricted entirely by the track 348.

The artificial intervertebral joint 300 may be installed between the vertebrae 7, 9 as described above. After installation, the spacer 332 may generally permit flexion-extension motion, anterior-posterior translation, and limited torsion. Lateral bending may be generally restricted although lift-off may be permitted. Coupling of flexion-extension and lateral bending motions may also be generally restricted. Any of these motions may be limited or altered by changing the length or direction of the track. In an alternative embodiment, the track may be omitted to restrict translation of the spacer, and the pin may permit only pivoting motion. In another alternative embodiment, the track may be curved or may arch in a sagittal plane. In another alternative embodiment, the spacer may be positioned such that it is elongated in an anterior-posterior direction and the track may extend in a lateral direction. In such an embodiment, lateral translation and lateral bending may be permitted and anterior-posterior translation and flexion-extension motion may be limited.

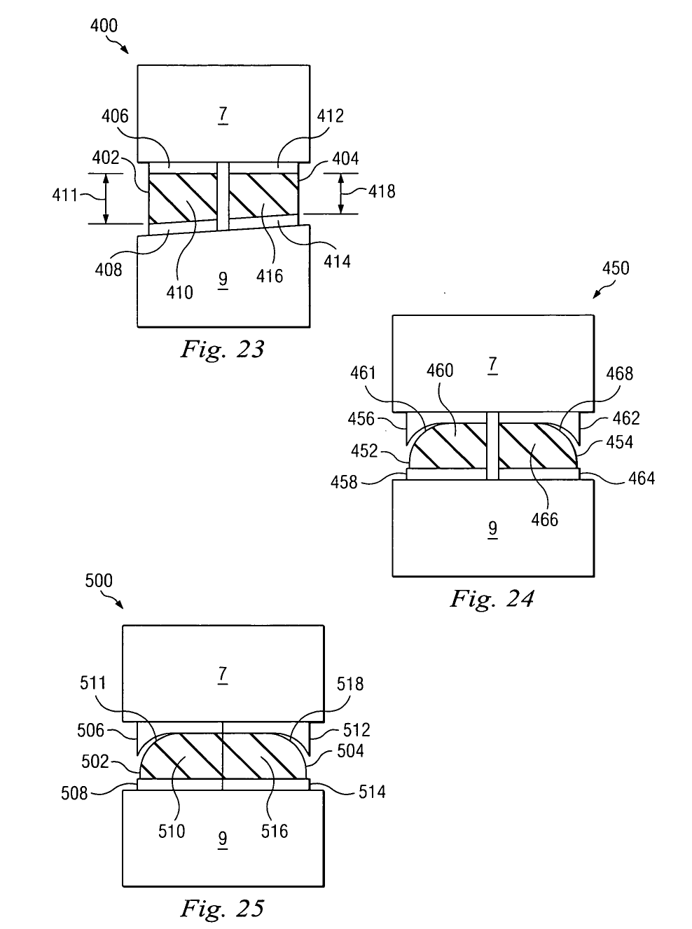

Referring now to FIG. 23, in this embodiment, an artificial intervertebral joint 400 may be substantially similar to the artificial intervertebral joint 200 except for the differences described. The joint 400 may comprise a pair of arthroplasty halves 402, 404. The arthroplasy half 402 may include a rostral anterior joint component 406 and a caudal anterior joint component 408 between which a spacer 410 may extend. The spacer 410 may have a rostral-caudal height 411. The arthroplasty half 404 may include a rostral anterior joint component 412 and a caudal anterior joint component 414 between which a spacer 416 may extend. The spacer 416 may have a rostral-caudal height 418. The height 411 may be larger than the height 418 to create a wedge effect between the vertebrae 7, 9, to correct alignment of the vertebrae 7, 9 or to create distraction. The heights of the spacers may be equal across the spacer, or the spacer itself may be tapered.

Referring now to FIG. 24, in this embodiment, an artificial intervertebral joint 450 may be substantially similar to the artificial intervertebral joint 200 except for the differences described. The joint 450 may comprise a pair of arthroplasty halves 452, 454. The arthroplasty half 452 may include a rostral anterior joint component 456 and a caudal anterior joint component 458 between which a spacer 460 may extend. The spacer 460 may have a curved lateral edge 461. The arthroplasty half 454 may include a rostral anterior joint component 462 and a caudal anterior joint component 464 between which a spacer 466 may extend. The spacer 466 may have curved lateral edge 468. The curved lateral edges 461, 468 may be rounded off about a common central axis. In this embodiment, the curved lateral edges 461, 468 may permit limited lateral bending in addition to the other types of motion described for intervertebral joint 200. Lift-off may still be permitted.

Referring now to FIG. 25, in this embodiment, an artificial intervertebral joint 500 may be substantially similar to the artificial intervertebral joint 450 except for the differences described. The joint 500 may comprise a pair of arthroplasty halves 502, 504. The arthroplasty half 502 may include a rostral anterior joint component 506 and a caudal anterior joint component 508 between which a spacer 510 may extend. The spacer 510 may have a curved lateral edge 511. The arthroplasty half 504 may include a rostral anterior joint component 512 and a caudal anterior joint component 514 between which a spacer 516 may extend. The spacer 516 may have curved lateral edge 518. The curved lateral edges 511, 518 may permit limited lateral bending in addition to the other types of motion described for intervertebral joint 200. In this embodiment, the spacers 510, 516 are engaged to form a unitized cylindrical mobile bearing with rounded lateral edges. The methods of engagement and similar unitized bearings are further described in U.S. Utility patent application Ser. No. (Attorney Docket No. P21756), filed on Jan. 7, 2005 and entitled “Split Spinal Device and Method.” This application has been incorporated by reference.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top, ” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

1. An artificial vertebral joint for interposition between a superior vertebra and an inferior vertebra, the artificial vertebral joint comprising:
a superior retaining portion;
an inferior retaining portion; and
a semi-cylindrical shaped mobile bearing adapted for insertion between the superior retaining portion and the inferior retaining portion, wherein the semi-cylindrical shaped mobile bearing is further adapted to move within the inferior retaining portion.

2. The artificial vertebral joint of claim 1 wherein the semi-cylindrical shaped mobile bearing is adapted for anterior-posterior translation within the inferior retaining portion.

3. The artificial vertebral joint of claim 1 wherein the semi-cylindrical shaped mobile bearing is adapted for lateral translation within the inferior retaining portion.

4. The artificial vertebral joint of claim 1 wherein the semi-cylindrical shaped mobile bearing is further adapted for rotation within the inferior retaining portion.

5. The artificial vertebral joint of claim 1 wherein the superior retaining portion comprises a semi-cylindrical shaped trough adapted to mate with the half-cylinder shaped mobile bearing.

6. The artificial vertebral joint of claim 5 wherein a longitudinal axis of the semi-cylindrical shaped trough is approximately parallel with a transverse axis extending through an intervertebral disc space between the superior and inferior vertebrae.

7. The artificial vertebral joint of claim 5 wherein a longitudinal axis of the semi-cylindrical shaped trough is approximately parallel with an anterior-posterior axis extending through an intervertebral disc space between the superior and inferior vertebrae.

8. The artificial vertebral joint of claim 1 wherein the semi-cylindrical shaped mobile bearing comprises a polished surface.

9. The artificial vertebral joint of claim 1 wherein the inferior retaining portion comprises a flattened surface bordered by a motion stop surface.

10. The artificial vertebral joint of claim 1 further comprising a pin connecting the semi-cylindrical shaped mobile bearing to the inferior retaining portion, wherein the semi-cylindrical shaped mobile bearing rotates about the pin.

11. The artificial vertebral joint of claim 10 wherein the inferior retaining portion comprises a track movably engaged with the pin.

12. The artificial vertebral joint of claim 11 wherein the track is parallel to an anterior-posterior axis through an intervertebral disc space between the superior and inferior vertebrae.

13. An artificial vertebral joint for interposition between a superior vertebra and an inferior vertebra, the artificial vertebral joint comprising:
a first arthroplasty half comprising a first superior retaining portion, a first inferior retaining portion, and a first semi-cylindrical shaped mobile bearing adapted for insertion between the first superior retaining portion and the first inferior retaining portion, wherein the first semi-cylindrical shaped mobile bearing is movable within the first inferior retaining portion, and
a second arthroplasty half comprising a second superior retaining portion, a second inferior retaining portion, and a second semi-cylindrical shaped mobile bearing adapted for insertion between the second superior retaining portion and the second inferior retaining portion, wherein the second semi-cylindrical shaped mobile bearing is movable within the second inferior retaining portion.

14. The artificial vertebral joint of claim 13 wherein the first and second arthroplasty halves are adapted for posterior insertion into an intervertebral disc space between the superior and inferior vertebrae.

15. The artificial vertebral joint of claim 13 wherein a first longitudinal axis of the first semi-cylindrical shaped mobile bearing is collinear with a second longitudinal axis of the second semi-cylindrical shaped mobile bearing.

16. The artificial vertebral joint of claim 15 wherein the first and second longitudinal axes are parallel to a transverse axis through the intervertebral disc space.

17. The artificial vertebral joint of claim 13 wherein the first and second longitudinal axes are approximately parallel with a transverse axis extending through an intervertebral disc space between the superior and inferior vertebrae.

18. The artificial vertebral joint of claim 13 wherein the first and second superior retaining portions comprise first and second semi-cylindrical shaped troughs, respectively and wherein the first and second semi-cylindrical shaped troughs are adapted to mate with the first and second semi-cylindrical shaped mobile bearings, respectively.

19. The artificial vertebral joint of claim 18 wherein a first longitudinal axis of the first semi-cylindrical shaped trough is collinear with a second longitudinal axis of the second semi-cylindrical shaped trough.

20. The artificial vertebral joint of claim 13 wherein the first semi-cylindrical shaped mobile bearing is adapted for anterior-posterior translation within the first inferior retaining portion.

21. The artificial vertebral joint of claim 13 wherein the first semi-cylindrical shaped mobile bearing is adapted for lateral translation within the first inferior retaining portion.

22. The artificial vertebral joint of claim 13 wherein the first semi-cylindrical shaped mobile bearing is further adapted for rotation within the first inferior retaining portion.

23. The artificial vertebral joint of claim 13 wherein the first semi-cylindrical shaped mobile bearing comprises a polished surface.

24. The artificial vertebral joint of claim 13 further comprising a first pin connecting the first semi-cylindrical shaped mobile bearing to the first inferior retaining portion, wherein the first semi-cylindrical shaped mobile bearing rotates about the first pin.

25. The artificial vertebral joint of claim 24 wherein the first inferior retaining portion comprises a track movably engaged with the pin.

26. The artificial vertebral joint of claim 25 wherein the first track is parallel to an anterior-posterior axis through an intervertebral disc space between the superior and inferior vertebrae.

27. The artificial vertebral joint of claim 25 wherein the first track is parallel to a transverse axis through an intervertebral disc space between the superior and inferior vertebrae.

28. The artificial vertebral joint of claim 13 wherein the first arthroplasty half further comprises a superior bridge connected to the first superior retaining portion and an inferior bridge connected to the first inferior retaining portion, and wherein the superior and inferior bridges extend from an intervertebral disc space between the superior and inferior vertebrae.

29. The artificial vertebral joint of claim 28 wherein the first arthroplasty half further comprises a superior posterior joint component connected to the superior bridge and an inferior posterior joint component connected to the inferior bridge, wherein the superior and inferior posterior joint components are movably engaged.

30. The artificial vertebral joint of claim 28 wherein the inferior bridge is at least a portion of an artificial pedicle.

31. The artificial vertebral joint of claim 13 wherein a first height of the first semi-cylindrical shaped mobile bearing is greater than a second height of the second semi-cylindrical shaped mobile bearing.

32. The artificial vertebral joint of claim 13 wherein a first lateral edge of the first semi-cylindrical shaped mobile bearing is curved and wherein a second lateral edge of the second semi-cylindrical shaped mobile bearing is curved.

33. The artificial vertebral joint of claim 13 wherein the first semi-cylindrical shaped mobile bearing is engaged with the second semi-cylindrical shaped mobile bearing to form a unitized bearing.

34. A method of implanting an artificial spinal joint, the method comprising:
creating first exposure through a patient's back to access an intervertebral space;
inserting at least a portion of the artificial spinal joint through the first exposure; and
positioning a first anterior joint portion of the artificial spinal joint in the intervertebral space, wherein the first anterior joint portion comprises a first superior retaining portion, a first inferior retaining portion, and a first semi-cylindrical shaped mobile bearing positioned between the first superior retaining portion and the first inferior retaining portion, wherein the first semi-cylindrical shaped mobile bearing is further adapted to move within the first inferior retaining portion.

35. The method of claim 34 further comprising:
positioning a posterior joint portion of the artificial spinal joint outside of the intervertebral space, wherein positioning a posterior joint portion includes engaging a posterior protrusion with a posterior socket.

36. The method of claim 34 further comprising:
creating a second exposure through the patient's back to access the intervertebral space and
positioning a second anterior joint portion of the artificial spinal joint in the intervertebral space, wherein the second anterior portion comprises a second superior retaining portion, a second inferior retaining portion, and a second semi-cylindrical shaped mobile bearing positioned between the second superior retaining portion and the second inferior retaining portion, wherein the second semi-cylindrical shaped mobile bearing is further adapted for translation within the second inferior retaining portion.

37. The method of claim 36 further comprising:
collinearly aligning a first longitudinal axis of the first semi-cylindrical shaped mobile bearing with a second longitudinal axis of the second semi-cylindrical shaped mobile bearing.

38. The method of claim 36 wherein the second exposure is on an opposite side of the patient's vertebral canal from the first exposure.

39. The method of claim 34 further comprising:
resecting at least one vertebral endplate to receive the first anterior joint portion.

40. The method of claim 34 further comprising:
resecting at least a portion of at least one natural facet.

41. The method of claim 34 wherein the step of positioning further comprises placing a first longitudinal axis of the first semi-cylindrical shaped mobile bearing in approximately parallel alignment with a transverse axis extending through the intervertebral space.

42. A system for creating at least a portion of a coupling between a superior vertebra and an inferior vertebra comprising:
a first means for connecting to the superior vertebra, the first means comprising a first retaining means;
a second means for connecting to the inferior vertebra, the second means comprising a second retaining means; and
a third means adapted for insertion between the first and second retaining means, wherein the third means comprises a semi-cylindrical shaped portion and wherein the third means is adapted to translate relative to the second retaining means.
